(19) United States
(12) Patent Application Publication
(10) Pub. No.: US 2023/0338648 A1
(43) Pub. Date: Oct. 26, 2023

(54) DEVICE FOR ADMINISTERING A MEDICAL LIQUID, AND CORRESPONDING METHOD

(71) Applicant: B. Braun Melsungen AG, Melsungen (DE)

(72) Inventors: Maria Buerger, Malsfeld (DE); Christoph Erlen, Kassel (DE); Jana Roeper, Kassel (DE)

(21) Appl. No.: 17/916,153

(22) PCT Filed: Jan. 11, 2022
(86) PCT No.: PCT/EP2022/050376

§ 371 Date: Sep. 30, 2022

(30) Foreign Application Priority Data
Jan. 15, 2021 (DE) 10 2021 100 818.2

(51) Int. Cl. A61M 5/50; A61M 5/145

(52) U.S. Cl. CPC A61M 5/145; A61M 5/5086; A61M 2205/12; A61M 2205/14; A61M 2205/27

(57) ABSTRACT

A device for administering a medical liquid and a method for operating the device. The device includes a main part having a receiving region and a drive. The receiving region can receive a liquid-containing hollow body. The drive can act on the hollow body such that liquid in the hollow body is conveyed. A control unit controls the drive. A cover is movably connected to the main part and covers the exterior of the receiving region in a closed position. The cover has a manually blockable input unit for inputting commands. In the closed position of the cover, a manual input can be entered on the input unit. A sensor detects whether the cover is in the closed position. The control unit ensures that the input unit is blocked when the sensor detects that the cover is open. Another device features a lockable cover.

### CROSS-REFERENCE TO RELATED APPLICATIONS

This application is the United States national stage entry of International Application No. PCT/EP2022/050376, filed Jan. 11, 2022, and claims priority to German Application No. 10 2021 100 818.2, filed Jan. 5, 2021. The contents of International Application No. PCT/EP2022/050376 and German Application No. 10 2021 100 818.2 are incorporated by reference herein in their entireties.

### FIELD

The present disclosure relates to a device for administering a medical liquid, and in particular to a syringe or infusion pump comprising a cover which is equipped with a manually blockable input unit and which is suitable for at least partly covering a syringe received in a receiving region of the syringe pump and, respectively, a tube received in a receiving region of the infusion pump in a closed position, and comprising a drive which is suitable for acting on a syringe received in the syringe pump and, respectively, on a tube received in the infusion pump such that a liquid contained in the received syringe and, respectively, in the received tube is conveyed. The present disclosure additionally relates to methods for operating devices for administering a medical liquid.

### BACKGROUND

A generic device for administering a medical liquid is disclosed, for example, in CN 211751505 U. The syringe pump according to CN 211751505 U has a main part which is equipped, on a front side in a position of use, with a receiving region for receiving a syringe. In addition, the device has a cover which is pivotably connected to the main part and which covers the receiving region in a closed position. On the cover a touch-sensitive screen is provided which, in the position of use of the device, extends on a front side when the cover is in its closed position. The device has a control unit. The control unit can be caused, using a key, to unlock the screen and the cover.

The problem of the device according to CN 211751505 U is that a key is required for secure operation thereof.

### SUMMARY

Therefore, it is the object of the present invention to provide a device for administering a medical liquid the operation of which can be simplified.

A device for administering a medical liquid according to the disclosure specifically is a syringe pump or an infusion pump. The device has a main part. The main part has a receiving region and a drive. The receiving region is suitable for at least partly receiving a liquid-containing hollow body. A hollow body in accordance with the present disclosure may be specifically a syringe, an infusion tube or any other tube of a medical system. The drive is suitable for acting on an at least partly received hollow body such that a liquid contained in the at least partly received hollow body is conveyed. In particular, the drive is capable of moving a plunger of a received syringe or of acting on a portion of a received tube by means of peristalsis.

For the control of the drive, the device includes a control unit.

The device has a cover which is movably, in particular pivotably, connected to the main part. In particular, a pivot axis of the cover extends in a position of use of the device at the front in a region of a lower edge of a housing of the device. The cover covers the exterior of the receiving region in a closed position.

“To cover” means in accordance with the present disclosure that a hollow body at least partly received in the receiving region of the device cannot be removed from the device without moving or pivoting the cover, at least not without damage.

A manually lockable input unit is provided on the cover. Via the input unit, commands can be input on the control unit.

The input unit can be particularly in the form of a manually lockable touch-sensitive screen (also known as touch screen or touch display). Alternatively, the input unit can also be designed as keys or the input unit can have keys in addition to a manually lockable touch-sensitive screen.

“Lockable” means in connection with the input unit within the scope of the disclosure that the input unit can be brought into a locked state in which the extent of the possible inputs is limited. In particular, the locked state can be configured such that merely an unlocking operation sequence can be input. An “operation sequence” in accordance with the present disclosure is understood to be a sequence of at least two inputs. To manually lock the input unit, a locking operation sequence can be provided. To unlock the input unit, said unlocking operation sequence can be provided.

If the input unit is designed as a touch-sensitive screen, the unlocking operation sequence can be configured such that, in a first step, the screen must be touched to trigger display of an information and, in a second step, in said information an appropriate surface must be pressed to unlock the screen.

As an alternative, or in addition, the device can be designed so that unlocking of the input unit can be accomplished using a, for example, electromechanical or capacitive push button which is separately arranged on the main part or on the cover or using plural, for example, electromechanical or capacitive push buttons which are separately arranged on the main part and/or on the cover.

The input unit is provided on the cover such that it is possible in the closed position of the cover to access at least a portion of the input unit from outside to perform a manual input on the input unit. In particular, the input unit is provided on the cover such that it extends, in the closed position of the cover, fully on an outside of the device, specifically on an outside in a position of use of the device at the front.

The device includes a sensor which is suitable for detecting whether the cover is in the closed position. The sensor can be a push button which reacts to touching caused by moving the cover. The sensor can be a position sensor which can detect the position of an electromechanical switch connected to the cover and the main part and, thus, can detect the position of the cover relative to the main part. The sensor can be a Hall sensor which reacts to moving a magnet provided in the cover. The sensor can be designed, within the scope of a light barrier, as a light-sensitive element.

The control unit is configured to determine, in the event of an opening the cover detected by the sensor, whether the input unit is blocked and, if the input unit is not blocked in the case of the detected opening of the cover, to block the input unit.

It is the advantage of the device according to the present disclosure that blocking of the input unit can be automatically ensured when the cover is opened.

According to one aspect of the disclosure, the control unit can be designed to unlock the input unit after opening the cover and thereby caused blocking of the input unit in the case of a closing of the cover detected by the sensor. Thus, it is possible to carry out, after temporarily opening the cover, that is, directly after closing the cover, manual inputs on the input unit without unlocking the input unit before manually, for example by means of a key.

In accordance with one aspect of the disclosure, the control unit can be designed to maintain, after a blocking of the input unit which was not caused by an opening of the cover, and after an opening of the cover in the case of a closing of the cover detected by the sensor, the blocking of the input unit, in particular to prevent inadvertent inputs. In other words, a blocking which is not caused by an opening, such as a blocking caused by manual input, can be prioritized. As described below, the device can be designed such that a blocking of the input unit is not only triggered by the opening of the cover or by manually inputting the blocking operation sequence, but also automatically after expiry of a predetermined period of time without any inputs on the input unit or by an excessive change of position of the device. In accordance with the present aspect of the disclosure, one of said other types of blocking of the input unit can be preferred such that an automatic unlocking when closing the cover is not accomplished. It is insignificant when the blocking which is not caused by the opening of the cover is carried out. For example, the input unit can be unlocked, after the opening and a related automatic blocking of the input unit, at first manually, such as by inputting the unlocking operation sequence, and then can be locked automatically, for example by omitting further inputs for a predetermined period of time. If then the cover is closed again, the input unit continues to be blocked according to the present aspect. Even if, already before an opening of the cover, the input unit is blocked, for example by inputting the blocking operation sequence, due to quite long inactivity or because of an excessive change of position of the device, a closing of the cover carried out after the opening of the cover causes no unlocking of the input unit according to the present aspect of the disclosure. Hence it is possible to prevent the input unit from being unintendedly unlocked.

In accordance with one aspect of the disclosure, the control unit can be designed to override or not to consider or to cancel, respectively, a manual input which is started before an opening of the cover and lasts during the opening of the cover. Thus, it is possible to seize the cover when manually opening the cover without consideration of any inadvertent inputs on the input unit. Accordingly, the difference between a single input and an input sequence should be noted. The device according to the disclosure can be designed such that the interaction of inputting an input sequence and blocking the input unit is as follows. When an input sequence consisting of plural individual inputs is started by inputting a first input and when the cover is opened, the blocking of the input unit initially prevents the other inputs of the input sequence from being input. When the input unit then is unlocked again by closing the cover, the input sequence can be continued to be input without having to repeat the already entered input. The present aspect of the disclosure, on the other hand, relates to the case that the cover is opened while a single input is entered, for example while a key of the input unit is pressed. Such an opening of the cover and the blocking of the input unit triggered in this way during entering one single input results in the fact, according to the present aspect of the disclosure, that the single input which has been started but not yet completed is discarded.

In accordance with one aspect of the disclosure, the device may include a sensor which is suitable for detecting whether a hollow body is received in the receiving region, and the control unit may be designed to perform an unlocking and/or locking of the input unit depending on the position of the cover only when a hollow body received in the receiving region is detected. Thus, the efficiency of the device can be increased.

In accordance with an alternative aspect, the present disclosure relates to a device for administering a medical liquid comprising a lockable cover. In particular, the device may be a syringe pump or infusion pump.

The device comprises a main part having a receiving region and a drive. The receiving region is suitable for at least partly receiving a liquid-containing hollow body. The hollow body may be a syringe, an infusion tube or any other tube of a medical system. The drive is suitable for acting on an at least partly received hollow body such that a liquid contained in the at least partly received hollow body is conveyed. In particular, the drive is capable of moving a plunger of a received syringe or of acting on a portion of a received tube by means of peristalsis.

The device comprises a control unit for controlling the drive.

In addition, the device comprises a cover which is movably, particularly pivotably, connected to the main part. In particular, a pivot axis of the cover extends in a position of use of the device at the front in a region of a lower edge of a housing of the device. The cover covers the exterior of the receiving region in a closed position.

On the cover a manually lockable input unit is provided. Commands can be input on the control unit via the input unit.

The input unit can particularly be designed in the form of a manually lockable touch-sensitive screen (also known as touchscreen or touch display). As an alternative, the input unit can also be designed in the form of keys or the input unit can include keys in addition to a manually lockable touch-sensitive screen. Incidentally, the foregoing remarks are also applicable, concerning the definitions and specifications of the obligatory and facultative features of the input unit of the device with the non-lockable cover, to the input unit of the device with a lockable cover.

The input unit is provided on the cover such that it is possible in the closed position of the cover to access at least a portion of the input unit from the outside to carry out a manual input on the input unit. In particular, the input unit is provided on the cover such that it extends in the closed position of the cover completely on an outside of the device, in particular on an outside in a position of use of the device at the front.

The device according to the alternative aspect comprises a locking device which is suitable for locking the cover in the closed position.

Moreover, the device comprises a sensor unit which is suitable for detecting whether the cover is locked in the closed position. For this purpose, the sensor unit can include two sensors one of which detects the position of the cover relative to the main part and the other detects the position of the locking device. The sensor relating to the cover can be designed, as afore-described, in the form of a push button that reacts to a touch caused by moving the cover or can be designed as a position sensor that can detect the position of an electromechanical switch connected to the cover and the main part and can detect thus the position of the cover relative to the main part, or can be designed as a Hall sensor that reacts to moving a magnet provided in the cover, or can be designed as a light-sensitive element within the scope of a light barrier. The sensor relating to the position of the locking device can be designed as a push button that reacts to a touch caused by a movement in the locking device. The sensor relating to the locking device can be designed as a Hall sensor that reacts to a movement of a magnet provided in the locking device, in particular in or on a movable latch. The sensor relating to the locking device can be implemented by means of a potentiometer which is connected to a movable latch. The sensor relating to the locking device can be designed as a light-sensitive element within the scope of a light barrier.

The sensor unit can also be implemented with the aid of only one sensor which detects the position of the latch connected to the main part relative to the cover so that the different states “closed and locked”, “closed and unlocked” and “opened and unlocked” are detected merely based on a signal. This can be implemented, for example, by the fact that a Hall sensor is provided in the cover and a magnet is provided in or on the latch of the locking device.

The control unit is designed to determine, in the case of unlocking of the closed cover detected by the sensor unit, whether the input unit is blocked, and, if the input unit is not blocked when the unlocking of the cover is detected, to block the input unit. In particular, the control unit and, resp., the device can be designed such that a command for unlocking the cover in the closed position entered particularly by means of a manual input via the input unit also causes an automatic opening of the cover.

The device according to the alternative aspect of the present disclosure advantageously protects the input unit from accidental inputs.

According to one aspect of the disclosure, the control unit can be designed to unblock the input unit after an unlocking and opening of the cover and a blocking of the input unit caused by unlocking in the case of a closing and locking of the cover detected by the sensor unit. Thus, after locking a cover which was unlocked before, a manual input can be carried out on the unblocked input unit without a user having to take any additional steps or measures.

According to one aspect of the disclosure, the control unit can be designed to maintain the blocking of the input unit after a blocking the input unit which was not caused by an unlocking of the cover, and after an unlocking of the cover in the case of a locking of the cover detected by the sensor unit. Thus, it is possible to improve the safety of the device. The term “blocking of the input unit not caused by unlocking the cover” refers, for example, to a manual blocking by inputting a blocking operation sequence, to automatic blocking after expiry of a predetermined period of time without any inputs on the input unit or to an automatic blocking due to a change of position of the device. According to the present aspect of the disclosure, it is insignificant whether the blocking of the input unit which is not caused by unlocking the cover is carried out before or after the unlocking of the cover.

According to one aspect of the disclosure, the control unit can be designed to cancel a manual input which was started prior to the unlocking of the cover and lasts when the cover is unlocked. Thus, it is possible to seize the cover when manually unlocking the cover without having to fear entering an unintended command. Accordingly, the already mentioned difference between individual inputs and input sequences consisting of individual inputs should be noted.

In accordance with one aspect of the disclosure, the device can comprise a sensor which is suitable for detecting whether a hollow body is received in the receiving region, and the control unit can be designed to carry out an unblocking and/or blocking of the input unit depending on the locking of the closed cover only when a hollow body received in the receiving region is detected. It is thus possible to increase the efficiency of the device.

In accordance with one aspect of the disclosure, the control unit can be designed to block the input unit after an unblocking and a subsequent expiry of a time interval in which no manual input is carried out on the input unit. Hence, it is possible to further improve the safety of the device.

According to one aspect of the disclosure, the device can comprise a position sensor which is suitable for detecting a change of position of the main part, and the control unit can be designed to determine, when the detected change of position exceeds a predetermined threshold, whether the input unit is blocked, and if the input unit is not blocked, to block the input unit. Thus, accidental entering of unintended commands on the input unit can be prevented during transport of the device.

As mentioned before, the input unit according to one aspect of the disclosure may comprise a touch-sensitive screen or may be designed as such. The screen can be designed as a resistive touchscreen, a capacitive touchscreen, a force-based touchscreen (piezo) or an ultrasound-based touchscreen.

The present disclosure further relates to a method for operating a device for administering a medical liquid.

The device to be operated is equipped with a main part which has a receiving region and a drive, the receiving region being suitable for at least partly receiving a liquid-containing hollow body, and the drive being suitable for acting on an at least partly received hollow body such that a liquid contained in the at least partly received hollow body is conveyed. A cover is movably connected to the main part. In a closed position, the cover covers the exterior of the receiving region, a manually blockable input unit for manually inputting commands on the control unit being provided on the cover such that, in the closed position of the cover, a manual input on the input unit is at least partly possible from outside.

The method for operating said device according to the disclosure comprises the steps of:
detecting whether the cover is in the closed position,
determining whether the input unit is blocked when the cover is detected to be opened, and
blocking the input unit, if the input unit is not blocked when the cover is detected to be opened.

The present disclosure further relates to a method for operating a device for administering a medical liquid comprising a lockable cover.

The device to be operated is equipped with a main part comprising a receiving region and a drive, the receiving region being suitable for at least partly receiving a liquid-containing hollow body, and the drive being suitable for acting on an at least partly received hollow body such that a liquid contained in the at least partly received hollow body is conveyed, with a cover movably connected to the main part which covers the exterior of the receiving region in a closed position, a manually blockable input unit for manually inputting commands on the control unit being provided on the cover such that in the closed position of the cover a manual input on the input unit is at least partly possible from outside, and with a locking device which is suitable for locking the cover in the closed position.

The method for operating said device according to the disclosure comprises the steps of:
detecting whether the cover is locked in the closed position,
determining whether the input unit is blocked when the closed cover is detected to be unlocked, and
blocking the input unit, if the input unit is not blocked when the cover is detected to be unlocked.

### BRIEF DESCRIPTION OF THE DRAWING FIGURES

In the following, the present invention will be described in detail based on preferred embodiments with reference to the attached drawings, wherein:

FIG. 1 shows a perspective view of a device according to a first embodiment of the present disclosure in the form of a syringe pump with a closed cover;

FIG. 2 shows a perspective view of the device shown in FIG. 1 with the cover being in an opened position;

FIG. 3 shows a perspective view of a device according to a second embodiment of the present disclosure in the form of an infusion pump with a closed cover;

FIG. 4 shows a perspective view of the device shown in FIG. 3 with the cover being in an opened position;

FIG. 5 shows a front view of the device shown in FIG. 4 according to arrow V in FIG. 4;

FIG. 6 shows a detail section view of a locking device according to arrow VI in FIG. 4 in a state just before a cover is closed and locked; and

FIG. 7 shows a detail section view of the locking device in a locked state.

### DETAILED DESCRIPTION

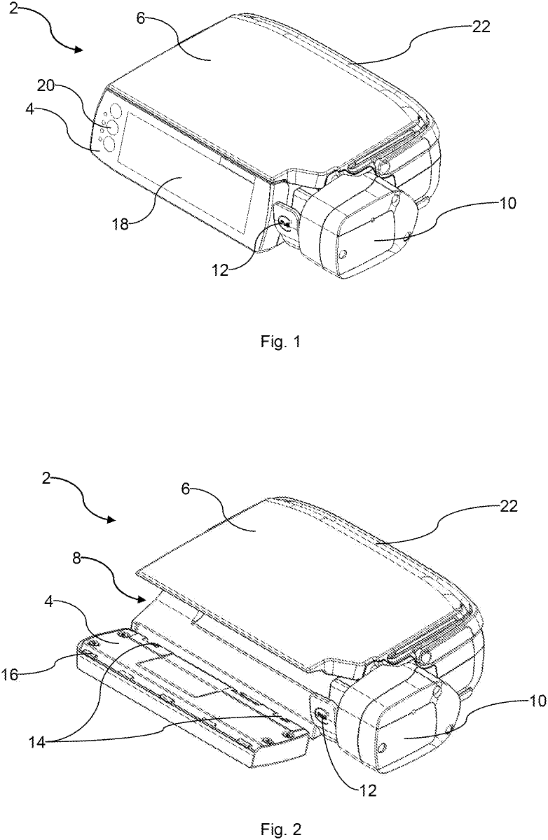

FIG. 1 and FIG. 2 illustrate perspective views of a device 2 according to a first embodiment of the present disclosure in the form of a syringe pump. In FIG. 1, the device 2 is shown with a cover 4 in a closed position. In FIG. 2, the device 2 is shown with the cover 4 in an opened position.

The device 2 has a substantially box-shaped main part 6. The device 2 includes the cover 4 on a long lateral surface of the main part 6 facing a user in the position of use of the device 2. The lateral surface of the main part 6 facing the cover 4 has a receiving region 8. The receiving region 8 is designed to receive a syringe (not shown) such that a plunger of the syringe can be displaced using a slide 10 disposed on a short side of the receiving region 8. The slide 10 is moved for this purpose by means of a drive integrated in the main part 6. To mount a syringe in the receiving region 8 in a simple manner, on the slide 10 a spring-loaded actuating element 12 is provided which, in an actuated state, detaches the slide 10 from the drive such that the slide 10 can be manually displaced.

The cover 4 is pivotably connected, via a hinge device 14, to a long edge of the main part 6 disposed at the bottom in the position of use of the device 2.

On an inside of the cover 4, spring clips 16 by means of which the cover 4 is clamped to the main part 6 in the closed position are provided at a long edge opposite to the hinge device 14.

On an outside of the cover 4 opposite to the inside, a lockable touch-sensitive screen 18 is provided which is connected to a control unit (not shown) provided in the main part 6. The screen 18 has the contour of an elongate rectangle and extends along the substantially rectangular cover 4. Plural buttons 20 connected to the control unit are provided at a short edge of the screen 18 opposite to the slide 10. The screen 18 and the buttons 20 form the input unit according to the disclosure in the present embodiment.

In the position of the cover 4 shown in FIG. 1, the screen 18 is usually unlocked. By manually inputting a blocking operation sequence on the screen 18, the control unit can block the screen 18. The blocking operation sequence comprises at least two steps to reduce a probability for inadvertent input of the blocking operation sequence. Accordingly, by manually inputting an unblocking sequence on the screen 18, the screen 18 can be unblocked. Also, the device can be designed so that at least one of the buttons 20 is included in the blocking operation sequence and/or the unblocking operation sequence, that is, must be pressed in the course of the respective sequence.

In the cover 4, at least one magnet (not shown) is provided. In the main part 6 at least one Hall sensor (not shown) connected to the control unit is provided.

When the cover 4 is pivoted out of the closed position shown in FIG. 1, the control unit detects a change of the signal of the Hall sensor and notes that the cover 4 is opened. The device can also be designed such that a push button/pressure sensor/contact sensor is installed in the cover 4 and/or in the main part 6, and that the control unit detects the closing and, correspondingly, the opening of the cover 4 based on the contacting of the push button/pressure sensor/contact sensor when the cover 4 is closed.

As soon as the control unit notes the cover 4 as opened, the control unit checks and, resp., determines whether the screen 18 is blocked. When the screen 18 is blocked already when the opened cover 4 is detected, the control unit leaves the screen 18 in the blocked state. When the screen 18 is not blocked when the opened cover 4 is detected, the control unit blocks the screen 18.

The device 2 includes a pivotable U-shaped carrying bracket 22. The free ends of the carrying bracket 22 are pivotably connected to the main part 6 centrally at each of the short edges on an upper side of the main part 6. In a folded position shown in FIG. 1 and FIG. 2, the carrying bracket 22 extends along the edges of the main part 6. In an unfolded position (not shown), the carrying bracket 22 is spread from the main part 6 and provides a handle for carrying the device 2.

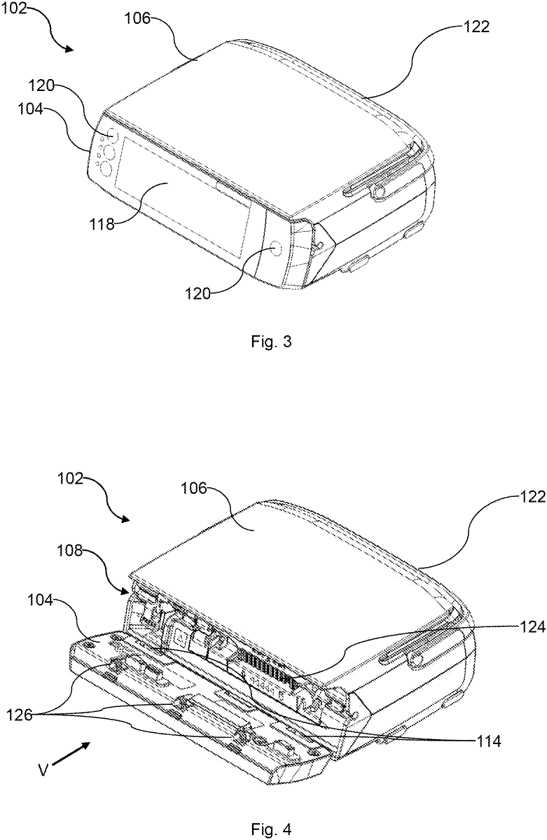

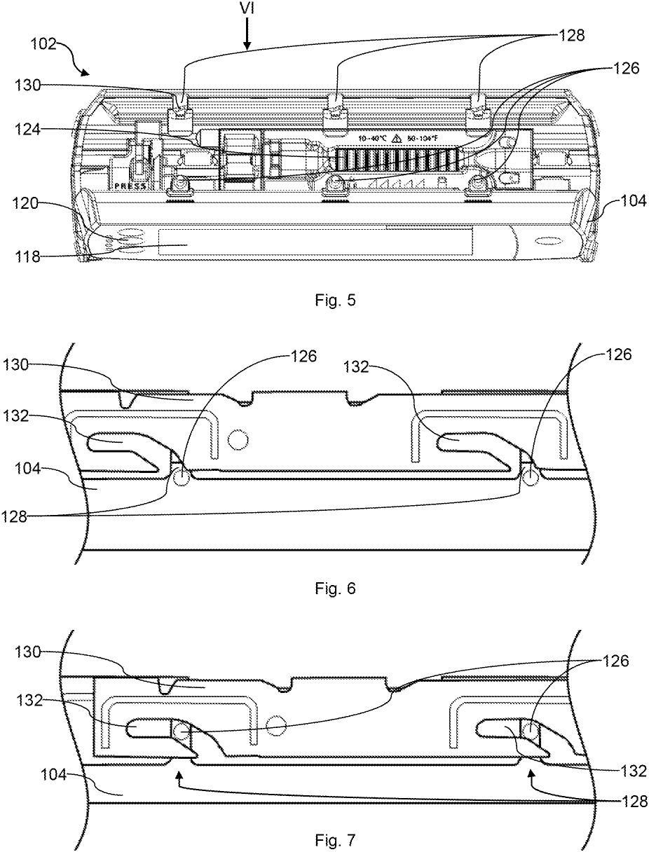

FIG. 3 and FIG. 4 illustrate perspective views of a device 102 according to a second embodiment of the present disclosure in the form of an infusion pump. FIG. 3 shows the device 102 with a cover 104 in a closed position. FIG. 4 shows the device 102 with the cover 104 in an opened position. FIG. 5 illustrates a front view of the device 102 with an opened cover 104. FIG. 6 and FIG. 7 illustrate section views of a part of a locking device for the cover 104 of the device 102 in an unlocked state (FIG. 6) and in a locked state (FIG. 7).

The device 102 includes a substantially box-shaped main part 106. On a long lateral surface of the main body 106 facing a user in the position of use of the device 102, the device 102 includes the cover 104. The lateral surface of the main part 106 facing the cover 104 has a receiving region 108. The receiving region 108 is designed to receive a tube (not shown) and includes a peristalsis 124 by means of which adjacent sections of the tube can be pinched off one after the other so that a liquid contained in the tube can be conveyed.

The cover 104 is pivotably connected, via a hinge device 114, to a long edge of the main part 106 disposed at the front bottom in the position of use of the device 102.

On an inside of the cover 104, at a long edge opposite to the hinge device 114 detent pins 126 are provided which are fitted, in the closed position of the cover 104, into corresponding receptacles 128 at an upper edge of the main part 106 in a position of use of the device 102 at the front and can be fixed in the receptacles 128 by means of a lock bar 130 (shown in FIGS. 5, 6 and 7) in order to lock the cover 104 in the closed position. As shown in FIG. 6 and FIG. 7, the lock bar 130 has a hook-shaped undercut 132 for each detent pin 126. In the closed position of the cover 104, the detent pins 126 are received in the corresponding receptacles 128. If the lock bar 130 is moved from the unlocked position into the locked position, the detent pins 126 are additionally received by the hook-shaped undercuts 132 of the lock bar 130 so that the receptacles 128 are blocked and the detent pins 126 are caught in the receptacles 128.

On an outside of the cover 104 opposite to the inside, a blockable touch-sensitive screen 118 is provided that is connected to a control unit (not shown) provided in the main part 106. The screen 118 has the contour of an elongate rectangle and extends along the substantially rectangular cover 104. At the short edges of the screen 118, plural buttons 120 connected to the control unit are provided. The screen 118 and the buttons 120 form the input unit according to the disclosure in the present embodiment.

In the position of the cover 104 shown in FIG. 3, the screen 118 is usually unblocked. By manually entering a blocking operation sequence on the screen 118, the control unit can block the screen 118. The blocking operation sequence comprises at least two steps to reduce a probability for an accidental input of the blocking operation sequence. Accordingly, by manually entering an unblocking sequence on the screen 118, the screen 118 can be unblocked. The device can also be configured such that at least one of the buttons 120 is included in the blocking operation sequence and/or the unblocking operation sequence, that is, must be pressed in the course of the respective sequence.

The lock bar 130 provided for fixing the detent pins 126 is coupled to a potentiometer (not shown) by means of which the control unit can determine the position of the lock bar 130 relative to the cover 104. The determination of the position of the cover 104 vis-à-vis the main part 106 can be implemented corresponding to the device 2, i.e., using a Hall sensor or using a push button/pressure sensor/contact sensor. Alternatively, the device 102 can also be designed such that the position of the lock bar 130 is determined corresponding to the position of the cover 104 using a Hall sensor, a push button or a light barrier. The potentiometer (not shown) and the sensor for determining the position of the cover (not shown) together constitute the sensor unit according to the disclosure.

In order to pivot the cover 104 from the closed position shown in FIG. 3 into the position shown in FIG. 4, a button predefined for this purpose among the buttons 120 must be pressed to cause the lock bar 130 provided for fixing the detent pins 126 to be displaced via the control unit. The control unit monitors the position of the cover 104 relative to the main part 106 and detects a change of the signal of the potentiometer and notes that the cover 104 is “locked and closed”, “unlocked and closed”, or “unlocked and opened”.

As soon as the cover 104 is noted by the control unit as unlocked, that is, as soon as the state of “locked and closed” is no longer present, the control unit checks whether the screen 118 is blocked. If the screen 118 is blocked already when the unlocked cover 104 is detected, the control unit leaves the screen 118 in the blocked state. If the screen 118 is not blocked when the unlocked cover 104 is detected, the control unit blocks the screen 118.

The device 102 includes a pivotable U-shaped carrying bracket 122. The free ends of the carrying bracket 122 are pivotably connected to the main part 106 on an upper side of the main part 106 centrally at each of the short edges. In a folded position shown in FIG. 3 and FIG. 4, the carrying bracket 122 extends along the edges of the main part 106. In an unfolded position (not shown) the carrying bracket 122 is spread from the main part 106 and provides a handle for carrying the device 102.

The above-described embodiments of the device according to the disclosure shown in FIGS. 1 to 7 constitute merely two possible implementations.

According to the described embodiments, the control unit is informed of the blocking operation sequence and the unblocking operation sequence by input on the screen 18 or 118. As an alternative, it is possible to input the blocking operation sequence and the unblocking operation sequence via the buttons 20 or 120. Alternatively, it is also possible to inform the control unit of the blocking operation sequence and the unblocking operation sequence partly by actuating at least one of the buttons 20 or 120 and partly by actuating at least one actuating field displayed on the screen 18 or 118.

The device 2 or 102 can be equipped with a sensor which is suitable for detecting an unfolding of the carrying bracket 22 or 122. The control unit can be designed to block the screen 18 or 118 when the carrying bracket 22 or 122 is unfolded.

1. A device for administering a medical liquid, the device comprising;
a main part which has a receiving region and a drive, the receiving region being configured for at least partly receiving a hollow body containing a liquid, and the drive being configured for acting on the hollow body such that the liquid is conveyed;
a control unit for controlling the drive;
a cover which is movably connected to the main part and which covers an exterior of the receiving region in a closed position;
an input unit that is manually blockable for manually inputting commands on the control unit, the input unit provided on the cover in an arrangement that allows a manual input to be entered on the input unit from outside the cover when the cover is in a closed position; and
a sensor configured for detecting whether the cover is in the closed position,
the control unit configured to determine whether the input unit is blocked when the sensor detects that the cover is opened, and to block the input unit when the input unit is not blocked and the cover is detected to be opened.

2. The device according to claim 1, wherein the control unit is configured to unblock the input unit after an opening of the cover that causes blocking of the input unit, when the sensor detects that the cover is closed.

3. The device according to claim 1, wherein the control unit is configured to maintain the input unit in a blocked condition after a blocking of the input unit, which is not caused by an opening of the cover and an opening of the cover when the sensor detects that the cover is closed.

4. The device according to claim 1, wherein the control unit is configured to cancel a manual input that is started prior to an opening of the cover and continues when the cover is opened.

5. The device according to claim 1, further comprising a sensor configured to detect whether the hollow body is received in the receiving region the control unit being configured to carry out unblocking and/or blocking of the input unit depending on a position of the cover only when the sensor detects that the hollow body is received in the receiving region.

6. A device for administering a medical liquid, the device comprising:
a main part which has a receiving region and a drive, the receiving region being configured for at least partly receiving a hollow body containing a liquid, and the drive being configured for acting on the hollow body such that the liquid is conveyed;
a control unit for controlling the drive;
a cover which is movably connected to the main part and covers an exterior of the receiving region in a closed position,
an input unit that is manually blockable for manually inputting commands on the control unit, the input unit being provided on the cover in an arrangement that allows a manual input to be entered on the input unit from outside the cover when the cover is in a closed position;
a locking device configured for locking the cover in the closed position; and
a sensor unit configured for detecting whether the cover is locked in the closed position,
the control unit configured to determine whether the input unit is blocked when the sensor unit detects that the cover is closed and unlocked, and to block the input unit when the input unit is not blocked and the cover is detected to be unlocked.

7. The device according to claim 6, wherein the control unit is configured to unblock the input unit after unlocking and opening of the cover and blocking of the input unit which is caused by unlocking, when the sensor unit detects that the cover is closed and locked.

8. The device according to claim 7, wherein the control unit is configured to maintain the input unit in a blocked condition after a blocking of the input unit, which is not caused by an unlocking of the cover and an unlocking of the cover when the sensor detects that the cover is locked.

9. The device according to claim 6, wherein the control unit is configured to cancel a manual input that is started before unlocking of the cover and continues when the cover is unlocked.

10. The device according to claim 6, further comprising a sensor configured for detecting whether the hollow body is received in the receiving region, the control unit configured to carry out an unblocking and/or blocking of the input unit depending on a locking of the cover that is closed only when the sensor detects that the hollow body is received in the receiving region.

11. The device according to claim 1, wherein the control unit is configured to block the input unit after unblocking and a subsequent expiry of a time interval in which no manual input is carried out on the input unit.

12. The device according to claim 1, further comprising a position sensor which is configured for detecting a change of position of the main part, the control unit is configured to determine, when the change of position exceeds a predetermined threshold, whether the input unit is blocked, and when the input unit is not blocked, to block the input unit.

13. The device according to claim 1, wherein the input unit includes a blockable touch-sensitive screen.

14. A method for operating a device for administering a medical liquid comprising a main part which has a receiving region and a drive, the receiving region being configured for at least partly receiving a hollow body containing a liquid, and the drive being configured for acting on the hollow body such that the liquid is conveyed, and comprising a cover which is movably connected to the main part and which covers an exterior of the receiving region in a closed position, and further comprising an input unit that is manually blockable for manually inputting commands on the control unit, the input unit being provided on the cover in an arrangement that allows a manual input to be entered on the input unit from outside the cover when the cover is in the closed position,
the method comprising the steps of:
detecting whether the cover is in the closed position;
determining whether the input unit is blocked when the cover is detected to be opened; and
blocking the input unit when the input unit is not blocked and when an opening of the cover is detected.

15. A method for operating a device for administering a medical liquid comprising a main part which has a receiving region and a drive, the receiving region being configured for at least partly receiving a hollow body containing a liquid, and the drive being configured for acting on the hollow body such that the liquid is conveyed, and comprising a cover which is movably connected to the main part and which covers an exterior of the receiving region in a closed position, and comprising an input unit that is manually blockable for manually inputting commands on the control unit, the input unit being provided on the cover in an arrangement that allows a manual input to be entered on the input unit from outside the cover when the cover is in the closed position, and further comprising a locking device which is configured for locking the cover in the closed position,
the method comprising the steps of:
detecting whether the cover is locked in the closed position;
determining whether the input unit is blocked when cover is detected to be closed and unlocked; and
blocking the input unit when the input unit is not blocked and when an unlocking of the cover is detected.
